(19) United States
(12) Patent Application Publication
(10) Pub. No.: US 2026/0341450 A1
(43) Pub. Date: Oct. 29, 2026

(54) METHOD FOR GENERATING DATABASE OF BODY STATUSES

(71) Applicant: LIMITED LIABILITY COMPANY DEVIMED, MOSCOW (RU)

(72) Inventor: Alexander Aleksandrovich KRAVCHENKO, Moscow (RU)

(21) Appl. No.: 19/274,363

(22) Filed: Jul. 18, 2025

(30) Foreign Application Priority Data
Apr. 26, 2025 (RU) 2025110912

(51) Int. Cl. G16H 20/10; G06F 3/0482; G07C 9/00; G16H 50/30

(52) U.S. Cl. CPC G16H 20/10; G06F 3/0482; G07C 9/00896; G16H 50/30

(57) ABSTRACT

The proposed invention relates to methods and means for identifying body statuses and can be used for distributing dosage forms depending on the body status. The technical problem to be solved by the proposed invention is to provide methods and/or products and/or implementations that make it possible to store and distribute dosage forms in their original packaging without opening it and to automatically access a dosage form and/or to signal that it is time to use a dosage form, including by means of a device for storing and distributing dosage forms.

### FIELD OF INVENTION

The proposed invention relates to methods and means for identifying body statuses and can be used for distributing dosage forms depending on the body status.

### DESCRIPTION OF THE RELATED ART

There are various methods and devices for storing and distributing dosage forms known in the art, such as, for example, the one disclosed in patent U.S. Pat. No. 11,433,000B2 (D1).

The common drawbacks of conventional methods and devices include the need to remove the dosage form from its original packaging in order to place it for storage in a device for storing and distributing dosage forms, which may adversely affect the storage conditions of the dosage form, thus, in turn, requiring said device to be equipped with specialized means to ensure that the storage conditions of the stored dosage form are correct. Yet another drawback is that conventional methods and devices cannot provide access to a dosage form in response to an identified body status.

There are various wearable user devices for identifying body status known in the art, such as smartwatches, fitness trackers, pulsimeters, heart rate monitors, etc., which are generally adapted to obtain a set of data about the body status, send them to a computing device and provide them to the user and/or attending physician; however, such data are generally limited to heart rate and a variety of body parameters that can be calculated based on the heart rate, and only rarely do said devices provide the user with data about certain substances present in the body.

The common drawbacks of conventional wearable user devices include the fact that it is impossible to use them in conjunction with a device for storing and distributing dosage forms so as to provide access to the stored dosage form in response to an identified body status, as well as the fact that it is impossible to provide the user and/or attending physician, depending on the treatment being applied and/or prescribed, with data about substances in a body that may prevent the usage of a medical drug.

Therefore, there is a problem of storing dosage forms in their original packaging in a device for storing and distributing dosage forms, as well as a problem of providing access to dosage forms stored in such a device in response to an identified body status. Yet another problem is that of providing the user and/or attending physician with data about substances in a body that may prevent the usage of a medical drug.

### SUMMARY OF THE INVENTION

The technical problem to be solved by the proposed invention is to provide methods and/or products and/or implementations that make it possible to store and distribute dosage forms in their original packaging without opening it and to automatically access a dosage form and/or to signal that it is time to use a dosage form, including by means of a device for storing and distributing dosage forms. In some aspects of the proposed invention, the technical problems also include the provision of methods and/or products and/or implementations that make it possible to access a stored dosage form in response to an identified body status. In some aspects of the proposed invention, the technical problems also include the provision of methods and/or products and/or implementations that make it possible to detect substances in a body that prevent the usage of a medical drug and/or dietary supplement. Another technical problem to be solved by the proposed invention is to expand the technical means, i.e., methods and/or products and/or implementations related to storage and distribution of dosage forms.

The objective of the proposed invention, in addition to it performing its functions, is to eliminate the drawbacks of the prior art and thus to ensure secure and safe storage of dosage forms in their original packaging without opening it in a device for storing and distributing dosage forms, as well as to provide automatic access to a dosage form and/or to automatically signal that it is time to use a dosage form, including by means of a device for storing and distributing dosage forms. In some aspects of the proposed invention, in addition to it performing its functions, the objectives also include automatic distribution of a dosage form in its original packaging in response to an identified body status. In some aspects of the proposed invention, in addition to it performing its functions, the objectives also include automatic notification of a user and/or attending physician about substances in a body that prevent the usage of a medical drug and/or dietary supplement.

The objective of the present invention is achieved by a method for generating a database of body statuses, the method executed by a processor of a computer device and comprising at least the following steps: at least by means of a graphical user interface of a user device, displaying a list of body statuses corresponding to one of a meal, physical activity, drug, or any suitable combination thereof; at least by means of a graphical user interface of a user device, selecting one body status from the list; by means of a wearable user device, obtaining at least one parameter of a body, namely one of a heart rate over a set period of time, a heart rate variability over a set period of time, presence of molecules of a certain substance in sweat fluids of a body within a set period of time, or any suitable combination thereof, wherein the parameter of the body is obtained when its body status corresponds to the selected one; based on the obtained parameter of the body, generating a dataset associated with the selected body status, wherein the dataset includes at least a sequence of measurements of the parameter of the body over a set period of time; generating a database of body statuses that includes at least one dataset associated with the selected body status; wherein a user device comprises at least: a processor of the user device; one of an input/output device of the user device or a combination of an input device of the user device and an output device of the user device; a transceiver of the user device; a memory of the user device that stores at least: a program code, which, when executed by the processor of the user device, causes a graphical user interface of the user device to be generated, at least adapted to render the list of body statuses and to select one body status from the list, a program code, which, when executed by the processor of the user device, causes the transceiver of the user device to receive the obtained parameter of the body, wherein a wearable user device comprises at least: a processor of the wearable user device; a measuring device, at least adapted to measure some parameter of the body during a set time period; a transceiver of the wearable user device; a memory of the wearable user device that stores at least: a program code, which, when executed by the processor of the wearable user device, causes the at least one parameter of the body to be obtained, and a program code, which, when executed by the processor of the wearable user device, causes the transceiver of the wearable user device to send the obtained parameter of the body.

### BRIEF DESCRIPTION OF THE DRAWINGS

Exemplary embodiments of the proposed invention are described in further detail below with references made to the attached drawings, included herein by reference:

FIG. 1 and FIG. 2 illustrate an exemplary, non-limiting, embodiment of the claimed device.

FIG. 3 illustrates an exemplary, non-limiting, embodiment of the claimed system.

FIG. 4 illustrates an exemplary, non-limiting, method for generating a database.

FIG. 5 and FIG. 6 illustrate an exemplary, non-limiting, embodiment of a wearable user device.

### DETAILED DESCRIPTION OF THE PREFERRED EMBODIMENTS

According to a preferred embodiment of the present invention, there is provided a method for generating a database of body statuses, the method executed by a processor of a computer device and comprising at least the following steps: at least by means of a graphical user interface of a user device, displaying a list of body statuses corresponding to one of a meal, physical activity, drug, or any suitable combination thereof; at least by means of a graphical user interface of a user device, selecting one body status from the list; by means of a wearable user device, obtaining at least one parameter of a body, namely one of a heart rate over a set period of time, a heart rate variability over a set period of time, presence of molecules of a certain substance in sweat fluids of a body within a set period of time, or any suitable combination thereof, wherein the parameter of the body is obtained when its body status corresponds to the selected one; based on the obtained parameter of the body, generating a dataset associated with the selected body status, wherein the dataset includes at least a sequence of measurements of the parameter of the body over a set period of time; generating a database of body statuses that includes at least one dataset associated with the selected body status; wherein a user device comprises at least: a processor of the user device; one of an input/output device of the user device or a combination of an input device of the user device and an output device of the user device; a transceiver of the user device; a memory of the user device that stores at least: a program code, which, when executed by the processor of the user device, causes a graphical user interface of the user device to be generated, at least adapted to render the list of body statuses and to select one body status from the list, a program code, which, when executed by the processor of the user device, causes the transceiver of the user device to receive the obtained parameter of the body, wherein a wearable user device comprises at least: a processor of the wearable user device; a measuring device, at least adapted to measure some parameter of the body during a set time period; a transceiver of the wearable user device; a memory of the wearable user device that stores at least: a program code, which, when executed by the processor of the wearable user device, causes the at least one parameter of the body to be obtained, and a program code, which, when executed by the processor of the wearable user device, causes the transceiver of the wearable user device to send the obtained parameter of the body.

According to an exemplary embodiment of the present invention, there is provided a method according to the present disclosure, characterized in that the measuring device is one of a pulsimeter, a transdermal sensor, or any suitable combination thereof.

According to an exemplary embodiment of the present invention, there is provided the disclosed method characterized in that said dataset is used for generating at least one instruction, according to which at least one control signal for unlocking a locking mechanism of a device for storing and distributing dosage forms is generated, wherein the device for storing and distributing dosage forms comprises at least: a compartment for storing a dosage form adapted to hold dosage forms in their original packaging, wherein the compartment is equipped with a processor-controlled locking mechanism; a processor adapted to control the locking mechanism; a transceiver; and a memory that stores at least: a program code, which, when executed, by a processor, causes at least one instruction to generate control signals for unlocking the locking mechanism to be received, and a program code, which, when executed by a processor, prompts it to generate a control signal for unlocking the locking mechanism in accordance with the at least one received instruction, wherein such instruction is generated using at least one dataset contained in the database generated using the above method for generating a database, performed by a processor of a computer device.

According to an exemplary embodiment of the present invention, there is provided a method according to the present disclosure, characterized in that the compartment is configured to hold dosage forms in their original packaging, such as one of the following: an ampoule, a jar, a bottle, a syringe, a box, a packet, a sachet, a tube, a contour bagless pack, a contour cellular (blister) pack, a vial, or a syringe tube.

According to an exemplary embodiment of the present invention, there is provided a method according to the present disclosure, characterized in that said instruction is received from a server of a system for distributing dosage forms.

According to an exemplary embodiment of the present invention, there is provided a method according to the present disclosure, characterized in that the memory additionally stores a program code, which, when executed by the processor, ensures generation and sending of a signal for unlocking the compartment.

According to an exemplary embodiment of the present invention, there is provided a method according to the present disclosure, characterized in that the compartment is equipped with at least one sensor adapted to detect the fact that the original packaging has been removed from the compartment.

According to an exemplary embodiment of the present invention, there is provided a method according to the present disclosure, characterized in that the compartment is equipped with at least one sensor adapted to detect the fact that the original packaging has been placed into the compartment.

According to an exemplary embodiment of the present invention, there is provided a method according to the present disclosure, characterized in that the memory additionally stores a program code, which, when executed by a processor, prompts it to generate a control signal for locking the locking mechanism.

According to an exemplary embodiment of the present invention, there is provided a method according to the present disclosure, characterized in that the control signal for locking the locking mechanism is generated in response to a detected fact of placing an original packaging into the compartment.

According to another preferred embodiment of the present invention, there is provided a method for identifying a body status, the method executed by a processor of a computer device and comprising at least the following steps: by means of a wearable user device, obtaining at least one parameter of a body, namely one of a heart rate over a set period of time, a heart rate variability over a set period of time, presence of molecules of a certain substance in sweat fluids of a body within a set period of time, or any suitable combination thereof, obtaining at least one body status associated with the obtained parameter of the body using a database of body statuses; identifying at least one body status associated with the obtained parameter of the body; wherein the database of body statuses is obtained using a method for generating a database of body statuses, the method executed by a processor of a computer device.

According to another preferred embodiment of the present invention, there is provided a method for generating instructions to generate control signals for a device for storing and distributing dosage forms, the method executed by a processor of a computer device and comprising at least the following steps: identifying a body status by means of a wearable user device; obtaining a dataset associated with the identified body status for generating an instruction to generate control signals; based on the obtained dataset, generating at least one instruction to generate control signals for a device for storing and distributing dosage forms.

According to another preferred embodiment of the present invention, there is provided a method for generating a notification to be displayed by an output device, the method executed by a processor of a computer device and comprising at least the following steps: identifying a body status by means of a wearable user device; obtaining a dataset associated with the identified body status; generating, based on the received dataset, at least one notification that is associated with at least a date and/or time.

According to another preferred embodiment of the present invention, there is provided a method for distributing dosage forms, the method executed by a processor of a computer device and comprising at least the following steps: identifying a body status by means of a wearable user device; obtaining a dataset associated with the identified body status; generating, based on the received dataset, at least one instruction to generate control signals; generating, based on the at least one generated instruction, at least one control signal for unlocking the locking mechanism of a device for storing and distributing dosage forms; and using the generated control signal to unlock a locking mechanism of the device for storing and distributing dosage forms.

According to other preferred embodiments of the present invention, there is provided a machine-readable storage medium (memory) that contains a program code, which, when executed by a processor of a computer device, prompts it to perform the steps according to any of the disclosed methods, at least partially.

According to other preferred embodiments of the present invention, there are provided computer devices comprising at least a memory and a processor, wherein the memory stores a program code, which, when executed by the processor of a computer device, prompts it to perform the steps according to any of the disclosed methods, at least partially.

According to other preferred embodiments of the present invention, there are provided systems comprising at least a user device that comprises at least a memory of the user device and a processor of the user device; a disclosed device for storing and distributing dosage forms; and a server that comprises at least a memory of the server and a processor of the server; wherein the user device is adapted to generate a query and send it to the server, in response to which the server generates a control signal for unlocking a locking mechanism and sends it to the device for storing and distributing dosage forms and/or sends an instruction to generate control signals to the device for storing and distributing dosage forms, and wherein the memory of the server stores a program code, which, when executed by a processor of a computer device, prompts it to perform the steps according to any of the disclosed methods, at least partially.

Additional alternative embodiments of the present invention are provided below. This disclosure is in no way limiting to the scope of protection granted by the present patent. Rather, it should be noted that the claimed invention can be implemented in different ways, so as to include different components and conditions, or combinations thereof, which are similar to the components and conditions disclosed herein, in combination with other existing and future technologies.

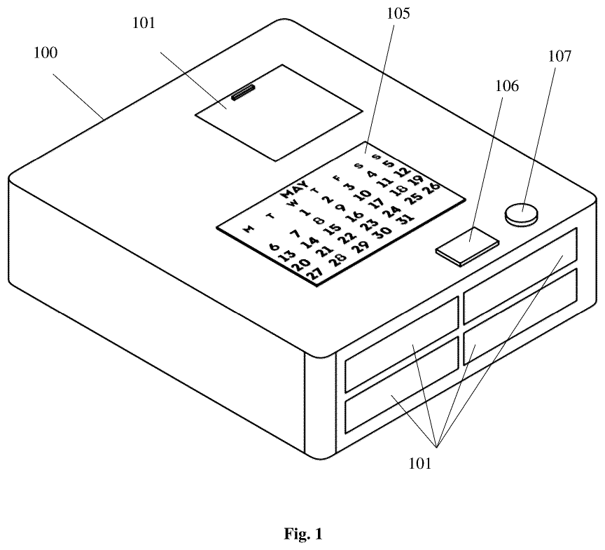

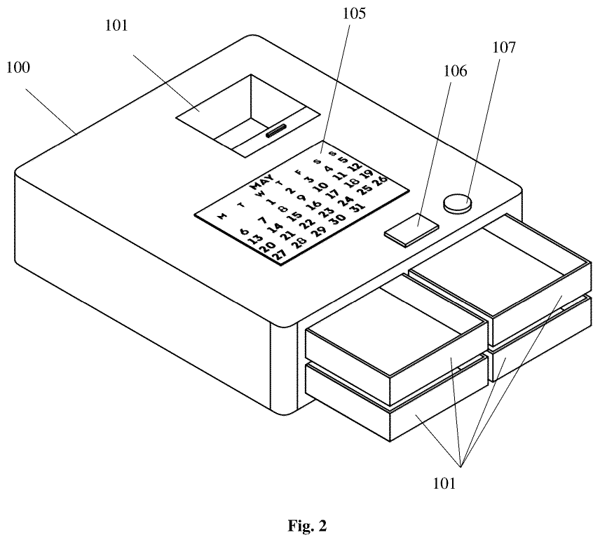

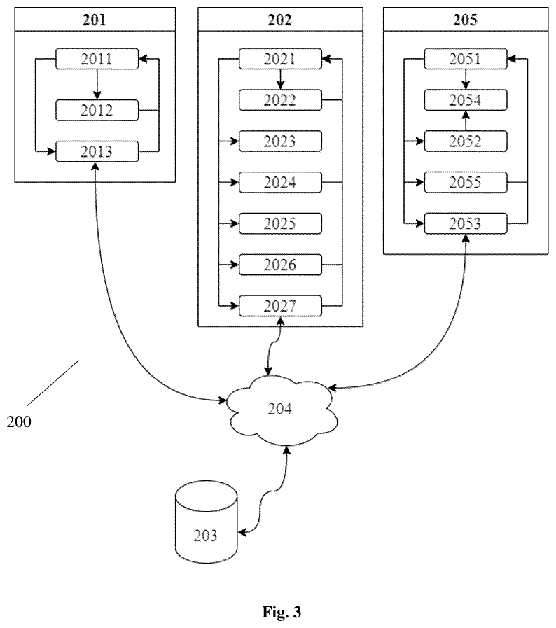

FIG. 1 and FIG. 2 illustrate an exemplary, non-limiting, embodiment of the claimed device 100 for storing and distributing dosage forms. Preferably, but not limited to, the claimed device 100 comprises at least: compartment 101 for storing a dosage form adapted to hold dosage forms in their original packaging, wherein compartment 101 is equipped with locking mechanism controlled by a processor; the processor adapted to control locking mechanism; a memory that stores at least a program code, which, when executed by processor, prompts it to generate a control signal for unlocking locking mechanism. For example, but not limited to, compartment 101 is configured to hold dosage forms in their original packaging, such as one of the following: an ampoule, a jar, a bottle, a syringe, a box, a packet, a sachet, a tube, a contour bagless pack, a contour cellular (blister) pack, a vial, a syringe tube, etc.; wherein, but not limited to, a dosage form is a medical drug in any form that corresponds to its original packaging; wherein, but not limited to, the term “dosage form” is provided in the present disclosure in the widest possible interpretation, comprising not only medical drugs or medicinal raw materials, but also non-medical drugs, such as, but not limited to, dietary supplements or other food that should be taken in accordance with instructions; and thus, preferably, but not limited to, it should be noted that the term “dosage form” in the present disclosure is used to mean any medical drugs, medicinal raw materials, non-medical drugs, such as dietary supplements, or other food that should be taken in accordance with instructions. For example, but not limited to, device 100 can be further equipped with a transceiver, wherein the memory additionally stores a program code, which, when executed by the processor, ensures reception of the control signal for unlocking locking mechanism or ensures reception of instructions to generate the control signal for unlocking locking mechanism. For example, but not limited to, the memory additionally stores a program code, which, when executed by the processor, prompts it to generate a control signal for unlocking locking mechanism, wherein, but not limited to, the control signal can be generated at set time or in response to instructions to generate the control signal, which, but not limited to, may be generated in response to an identified body status and/or when a certain time arrives, as will be described in more detail below; wherein, but not limited to, a body is a body of a person, such as the user of device 100, including a patient, and/or, but not limited to, an animal which has to take the dosage form stored in device 100. For example, but not limited to, device 100 can be equipped with the transceiver, wherein the memory additionally stores a program code, which, when executed by the processor, ensures reception of a control signal for unlocking locking mechanism and/or instructions for generating said control signal from server 201 of system 200 (see FIG. 3) for distributing dosage forms, wherein, but not limited to, the control signal can be generated at set time, which, but not limited to, may be provided by server 201, and/or in response to instructions from server 201 to generate the control signal, which, but not limited to, may be generated in response to an identified body status, received by server 201, and/or when a certain time arrives, as will be described in more detail below.

For example, but not limited to, the memory additionally stores a program code, which, when executed by the processor, ensures generation and sending of a signal for unlocking the compartment, for example, but not limited to, to user device 202 of system 200 and/or server 201 of system 200, whereby, for example, but not limited to, a signal regarding the availability of a dosage form is ensured, which, for example, but not limited to, may ensure timely notification of an administrator of system 200 that there has been an unauthorized access to the dosage form, which, for example, but not limited to, may prove necessary in case access to device 100 and, accordingly, to dosage forms stored therein should be restricted, for example, but not limited to, when device 100 can be interacted with by a child, an elderly person, or any other user that doesn't have full control over their actions.

For example, but not limited to, compartment 101 may also be equipped by at least one sensor adapted to detect the fact that the original packaging has been removed from compartment 101, wherein, for example, but not limited to, sensor may be one of the following: a video camera, a weight sensor, a motion sensor, a presence sensor, etc. or any suitable combination thereof; in addition, for example, but not limited to, the motion sensor and presence sensor are ultrasound sensors that are chosen so as not to affect the dosage form in any significant way; in addition, for example, but not limited to, the video camera is configured to record footage in poor lighting and/or absence of light. For example, but not limited to, sensor, in addition to or instead of detecting the fact that the original packaging has been removed, may detect the fact that the original packaging has been placed into compartment 101, wherein, for example, but not limited to, sensor is used to do that. For example, but not limited to, in response to removing an original packaging from compartment 101, a removal signal can be generated, which can be sent by means of the transceiver to server 201 and/or user device 202, thereby, for example, but not limited to, ensuring timely notification of the fact that a dosage form has been removed for use; in addition, for example, but not limited to, there may be set a certain time interval, within which the dosage form has to be returned to compartment 101, which can be detected by sensor, whereupon a placement signal can be generated, which can be then sent to server 201 and/or user device 202 to indicate that the use of the dosage form has ended; in addition, but not limited to, if a placement signal has not been generated within said time interval, a reminder signal can be generated, which can then be sent to server 201 and/or user device 202, to indicate that the dosage form has to be returned to compartment 101. For example, but not limited to, the placement signal can also be generated when a dosage form is first placed into compartment 101; wherein, but not limited to, in response to the generated placement signal, sensor can detect at least one property of the dosage form that would allow to identify its original packaging and, accordingly, the dosage form itself, wherein, for example, but not limited to, such properties may include a text, a marking, the shape of a solid dosage form, the weight of the original packaging and other unique properties which make it possible to identify the dosage form; for example, but not limited to, a video camera can provide the image of the original packaging, which can be recognized using a machine learning model trained using a suitable dataset that includes images of original packagings, or, for example, but not limited to, by means of comparing images of original packagings using a database, wherein a correspondence between the image of the original packaging and the dosage form can be provided, and wherein, but not limited to, multiple video cameras can be used to improve the detection accuracy, whereby enough images of the original packaging are obtained to accurately identify the dosage form; for example, but not limited to, a weight sensor can provide the weight of the original packaging, and a correspondence between the weight and the dosage form can be predicted, for example, using a machine learning model trained using a suitable dataset that includes weights of original packagings; for example, but not limited to, a motion sensor and/or presence sensor can provide a signature of the original packaging, and a correspondence between the signature and the dosage form can be predicted, for example, using a machine learning model trained using a suitable dataset that includes signatures of original packagings that have been detected by the motion sensor and/or presence sensor; wherein, for example, but not limited to, the machine learning model can be one of a classification model or a clustering model; wherein, for example, but not limited to, the classification model is based on one of: logistic regression, support vector machine, decision tree learning, random forest, naive Bayes classifier, k-nearest neighbors, neural network, boosting, gradient boosting, bagging, the method of group consideration of arguments, or any suitable combination thereof; and wherein, for example, but not limited to, the clustering model is based on one of: k-means, Density-based spatial clustering of applications with noise (DBSCAN), hierarchical clustering, spectral clustering, or any suitable combination thereof. For example, but not limited to, a dosage form can be identified using the processor, in which case the memory may store a corresponding program code, a machine learning model and/or a corresponding database that contains properties of original packagings. For example, but not limited to, a detected property of the original packaging can be sent via the transceiver to server 201, wherein the dosage form can be identified using processor 2011 of server 201, in which case memory 2012 of server 201 (see FIG. 3) may store a corresponding program code, a machine learning model and/or a corresponding database that contains properties of original packagings. For example, but not limited to, a detected property of the original packaging can be sent via the transceiver to user device 202, wherein the dosage form can be identified using processor 2021 of user device 202, in which case memory 2022 of user device 202 (see FIG. 3) may store a corresponding program code, a machine learning model and/or a corresponding database that contains properties of original packagings.

For example, but not limited to, device 100 can be additionally equipped with display 105, which is linked to the processor, to display information related to the functioning of device 100, for example, but not limited to, information regarding storage and/or distribution of dosage forms, and/or, for example, but not limited to, information regarding body status; for example, but not limited to, a month or part of a month of a suitable calendar, for example, but not limited to, the Gregorian calendar, can be rendered, indicating the current day and/or one or more days when the dosage form is to be distributed; for example, but not limited to, the degree of filling of compartments 101 can be rendered; for example, but not limited to, the body status can be rendered; for example, the current time, date, and/or time before the stored dosage form is to be distributed can be rendered; for example, but not limited to, display 105 can be a sensor display that can be used to control device 100 and/or generate instructions and commands to be sent to server 201 and/or user device 202, wherein, for example, but not limited to, control elements can be rendered to be interacted by the user in order to generate instructions and commands prompting the processor to execute at least the procedures, instructions and commands disclosed herein, which, accordingly, will not be described any further. For example, but not limited to, locking mechanism of at least one compartment 101 can be a biometric lock adapted at least to obtain biometric data associated with the body of a patient or with another user that can be used to authenticate them in order to control access to one or more compartments 101; in addition, for example, but not limited to, biometric data can be obtained using one or more biometric sensors 106 placed in such a way so as to facilitate interaction between the user or patient and biometric sensor 106, which may be represented by one of a fingerprint scanner, an iris scanner, a facial recognition scanner, a voice recognition scanner, a heart rate scanner, or any suitable combination thereof; in addition, for example, but not limited to, the authenticating biometric data can be obtained by means of an external device linked to device 100, for example, but not limited to, by user device 202, which, accordingly, can be equipped with at least one biometric sensor 2023 used to obtain the authenticating biometric data to authenticate the user of device 100.

For example, but not limited to, sensor display 105 can be used to control access to compartment 101, in which case display 105 can render control elements for inputting any authenticating non-biometric information, for example, but not limited to, a graphic code, PIN code, or other authenticating non-biometric data. For example, but not limited to, user authentication using authenticating non-biometric information, for example, but not limited to, a graphic code, PIN code, or other authenticating non-biometric data, can also be performed using an external device linked to device 100, such as, for example, but not limited to, user device 202, which, accordingly, can be equipped with at least one input device 2024, which can also be an input/output device, for example, but not limited to, a sensor display used to obtain authenticating non-biometric information to authenticate the user of device 100, whereupon an access signal can be generated and sent to device 100 or server 201, after which said access signal and/or a separate access signal is sent to device 100 in order to generate a control signal for unlocking locking mechanism; wherein, for example, but not limited to, in case input device 2024 is not an input/output device, user device 202 can be further equipped with output device 2025. For example, but not limited to, device 100 can be equipped with at least one independent control means 107 that generates control signals for the components of device 100, for example, but not limited to, for at least one of the following functions: selection of compartment 101 for interaction, unlocking of locking mechanism, setup of device 100, turning device 100 on or off, etc., or any suitable combination thereof; wherein, for example, but not limited to, control means 107 can be implemented as one or more control buttons. For example, but not limited to, compartment 101 can additionally be thermally insulated to ensure secure long-term storage of a dosage form in its original packaging; in addition, for example, but not limited to, compartment 101 can be thermally insulated by removing the heat generated by the heat-producing components of device 100, such as, for example, but not limited to, the power supply unit, in which case, for example, but not limited to, a radiator-based or fan-based heat removal system can be provided; at the same time, for example, but not limited to, compartment 101 can be thermally insulated by dividing the space inside the housing of device 100 using at least one heat-insulating partition; at the same time, for example, but not limited to, compartment 101 can be thermally insulated by enhancing the walls of compartment 101 with a suitable heat-insulating material, wherein, in case compartment 101 is mobile, its walls can be enhanced with a suitable heat-insulating material so as not to restrict the free movement of compartment 101, which, for example, but not limited to, may be achieved by enhancing the internal walls of compartment 101 with a suitable heat-insulating material; at the same time, for example, but not limited to, compartment 101 can be thermally insulated by making at least part of the housing of device 100 thermally insulated, which, for example, but not limited to, may be achieved by enhancing the internal walls of the housing of device 100 with a suitable heat-insulating material, wherein, but not limited to, in case the walls of compartment 101 are enhanced with a heat-insulating material and, accordingly, the housing of device 100 has a lid for compartment 101 (not shown in the drawings), said lid can also be enhanced with a heat-insulating material and/or can be made of a heat-insulating material, wherein, for example, but not limited to, said lid can be made transparent to provide visual access to the stored dosage form; for example, but not limited to, it can be made of a fire resistant glass or fire resistant glass-ceramics, etc.

For example, but not limited to, device 100 is implemented as a desktop device, i.e., its overall dimensions are chosen so that device 100 is easily usable in everyday life, placed, for example, on a dining table or near a drinking water source, and, therefore, is easily usable, for example, in a hospital ward. In addition, but not limited to, device 100 can be implemented as a wending machine or a display case that can be placed, for example, in a healthcare facility providing inpatient care to be used, for example, by a large number of people; in this case, for example, but not limited to, device 100 can be connected to a server of a medical system for distributing dosage forms, the administrator of which provides patients with access to the dosage forms stored in such a device.

For example, but not limited to the processor can be either a processor proper or a coprocessor and can be represented by one of the following: a CPU, a GPU, a C-RAM, an NPU, a QPU, a multiprocessor, or any suitable combination thereof; wherein, for example, but not limited to, the processor or coprocessor can be implemented as a controller or microcontroller; wherein, for example, but not limited to, the processor or coprocessor can be single-core or multicore; wherein, for example, but not limited to, the processor or coprocessor can be represented by a single processor or coprocessor or by any suitable combination of multiple processors and/or coprocessors; in addition, for example, but not limited to, it should be noted that the processor is chosen so as to ensure generation and/or execution of procedures, instructions, commands, control signals disclosed herein, which, accordingly, will not be described any further. For example, but not limited to, any of memories (machine-readable storage media) may comprise a non-volatile memory (NVRAM), a random-access memory (RAM); a read-only memory (ROM), an electrically erasable programmable read-only memory (EEPROM), a flash drive, a solid-state drive, or other memory technologies, a CD-ROM, a digital versatile disk (DVD) or other optical/holographic media, magnetic tapes, magnetic film, a hard disk drive or any other magnetic drive, and any other medium capable of storing and encoding the necessary information. In addition, but not limited to, the memory comprises a machine-readable storage medium based on the memory, either volatile or non-volatile, or a combination thereof. In addition, but not limited to, exemplary hardware devices include solid-state drives, hard disk drives, optical disk drives, etc. For instance, but not limited to, the computer-readable medium (memory) is not a temporary memory (i.e., a permanent, non-transitive memory), and therefore it does not contain a temporary (transitive) signal. In addition, but not limited to, the memory may store an approximate environment, in which computer instructions or codes, including those stored in the memory, can be used to execute a control procedure for device 100, such as generation of a scenario for locking or unlocking compartment 101, detection of removal, placement, presence of a dosage form, or detection of dosage form properties. In addition, but not limited to, system 200 may further comprise database 203. Database 203 may be, but not limited to, a hierarchical database, a network database, a relational database, an object database, an object-oriented database, an object-relational database, a spatial database, a combination of two or more said databases, etc. In addition, but not limited to, database 203 may store pre-recorded control procedures for device 100, as well as data for analysis and recognition, and other information, procedures, methods, means, instructions, etc. in the memory or a suitable memory of a different computer device connected to device 100 and/or user device 202 and/or server 201, which may be, but not limited to, identical to any of memories, as was disclosed above, and which can be accessed via data exchange network 204. In addition, but not limited to, the memory that stores information, procedures, methods, means, commands, and databases disclosed herein, can be distributed, i.e. implemented using a plurality of hardware devices, optionally integrated into a single computer device; thus, for example, but not limited to, individual parts of program codes, which, when executed by a processor, prompts it to perform the steps of any computer processor-performed method of the present disclosure, can be stored in separate hardware devices that are interconnected, including via data exchange network 204, and therefore such individual parts of program codes can be executed by different processors. In addition, but not limited to, there is provided a server 201, which, in addition to the functions mentioned above, is capable of storing and facilitating the execution of computer-readable commands and codes disclosed herein, which, accordingly, won't be described again. In addition, but not limited to, server 201, in addition to the functions mentioned above, is capable of controlling data exchange in system 200. In addition, but not limited to, data exchange within system 200 is performed with the help of one or more data exchange networks 204. In addition, but not limited to, data exchange networks 204 may include, but not limited to, one or more local area networks (LAN), optionally including, but not limited to, one or more personal area networks (PAN), and/or wide area networks (WAN), or may be represented by the Internet or Intranet, or a virtual private network (VPN), or a combination thereof, etc.; in addition, but not limited to, network 204 can be based on any suitable technology, for example, but not limited to, NFC, RFID, Beacon technology, or any suitable combination thereof, etc.; and it can use electromagnetic radiation (EMR) of any suitable wavelength and any suitable power for data exchange, for example, but not limited to, infrared, visible radiation (visible spectrum), radio waves, X-rays, or any suitable combination thereof, etc.; in addition, but not limited to, the devices of system 200 can communicate via transceivers, namely transceiver 2013 of server 201, transceiver 2027 of user device 202, the transceiver of device 100, and transceiver 2053 of wearable user device 205. In addition, but not limited to, server 201 is further capable of providing a virtual computer environment for the components of system 200 to interact with each other. In addition, but not limited to, network 204 enables interaction between device 100, user device 202 and/or server 201 and, optionally, database 203. In addition, but not limited to, database 203 can be implemented in the memory. In addition, most typically, but not limited to, the components of device 100, user device 202 and server 201 are interconnected, including via any kind of data bus. In addition, most typically, but not limited to, the components of device 100, user device 202, server 201 and wearable user device 205 can be powered by any suitable power source, such as electric grid, which can be accessed, for example, but not limited to, by a power supply unit (a plug), or an energy storage device (a storage battery). In addition, most typically, but not limited to, at least the components of device 100 are integrated into a single housing. In addition, most typically, but not limited to, user device 202 can be one of the following: a portable user device, a stationary user device, or a wearable user device 205; wherein, for example, but not limited to, a portable user device can be one of a remote control, a smartphone, a PDA, a tablet, a laptop, a netbook, etc.; wherein, for example, but not limited to, a stationary user device can be one of a PC, or a treadmill, a stationary bicycle, an elliptical trainer, a multifunctional training station, etc., equipped with a computer device and/or body status monitoring system; wherein, for example, but not limited to, a wearable user device can be one of a fitness tracker, a smartwatch, a heart rate monitor, smart glasses, smart textiles, implantable devices, etc.

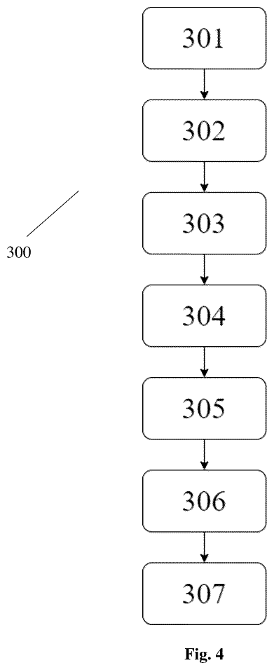

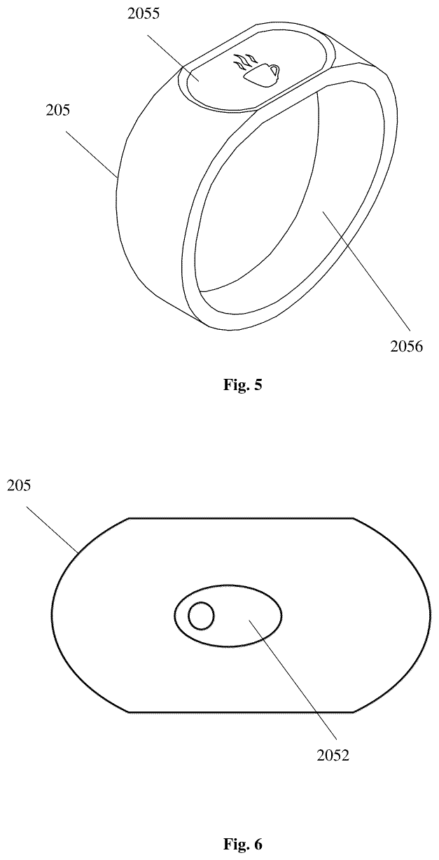

In addition, as shown in FIG. 4, there may be provided a method for generating a database, the method executed by a processor of a computer device and comprising at least the following steps: obtaining 301 at least a plurality of sources of official information about the medical drug and assigning a unique identifier to each source; from each obtained source of official information about the medical drug, extracting 302 at least an uncleaned entry identifying the dosage form of the medical drug, an unprocessed entry identifying the dosage regimen of the medical drug, an unprocessed entry identifying the method of administration of the medical drug; cleaning 303 each extracted entry identifying the dosage form of the medical drug in order to obtain corresponding cleaned entries identifying the dosage form of the medical drug and assigning to each the unique identifier of the source of official information about the medical drug, from which the uncleaned entry that was used to obtain the cleaned one had been extracted; semantically and syntactically analyzing 304 each extracted entry identifying the dosage regimen of the medical drug in order to obtain for each such entry at least an entry identifying the maximum recommended single dose of the medical drug, an entry identifying the maximum recommended daily dose of the medical drug, an entry identifying the standard course of administration of the medical drug; semantically and syntactically analyzing 305 each extracted entry identifying the method of administration of the medical drug in order to obtain for each such entry at least an entry identifying the method of administration of the medical drug, an entry identifying the administration of the medical drug relative to meals; assigning 306 to each semantically and syntactically analyzed entry the unique identifier of the source of official information about the medical drug, from which the unprocessed entry that was analyzed had been extracted; generating 307 a database containing at least a plurality of unique identifiers, each corresponding to a dataset that contains at least a cleaned entry identifying the dosage form of the medical drug, an entry identifying the maximum recommended single dose of the medical drug, an entry identifying the maximum recommended daily dose of the medical drug, an entry identifying the standard course of administration of the medical drug, an entry identifying the method of administration of the medical drug, an entry identifying the administration of the medical drug relative to meals; wherein, for example, but not limited to, the steps of method 300 do not have to be performed in sequence; if necessary, they may be performed simultaneously or in a different order from that disclosed herein. For example, but not limited to, the dataset obtained using method 300 is required for generating at least one instruction, in accordance with which at least one control signal for unlocking the locking mechanism of device 100 is generated. For example, but not limited to, the dataset obtained using method 300 is used to generate at least one notification to be shown on the display of user device 202, which is obtained by means of a program code executed by processor 2021 of said user device 202, which provides at least the following: an entry is generated, which is associated at least with a date and/or time, wherein the entry is generated using at least one dataset contained in the database generated using method 300; and the entry is shown on the display of the user device when the date and/or time it is associated with arrives, in which case, but not limited to, user device 202 can be equipped with input/output device 2024 (a sensor display).

For example, but not limited to, after a database has been generated using method 300, there may be generated a query to this database, containing at least the unique identifier of the source of official information about the medical drug. For example, but not limited to, in response to such a query, a dataset can be obtained that is associated with the source of official information about the medical drug whose unique identifier was included in the database query; wherein, for example, but not limited to, the dataset is not necessarily stored in the database that was generated using method 300, which, accordingly, contains at least only the unique identifiers associated with datasets, but it may be stored in a different database, i.e. upon receiving the query containing a unique identifier, a subsequent query for the required dataset can be generated to a corresponding database that stores datasets.

For example, but not limited to, the obtained dataset can be used to generate at least one instruction to generate control signals for unlocking the locking mechanism of device 100. For example, but not limited to, such an instruction may contain at least the time at which to generate the control signal for unlocking the locking mechanism of device 100. Thus, for example, but not limited to, there may be provided a method for distributing dosage forms, comprising at least the following steps: receiving at least one instruction to generate control signals for unlocking the locking mechanism of device 100, and upon receiving such an instruction, generating at least one control signal for unlocking the locking mechanism of device 100.

For example, but not limited to, the source of official information about the medical drug is one of the following: a general description of the medical drug (GDMD), a prescribing information about the medical drug (PI), or a similar suitable official document; wherein, but not limited to, it should be generally noted that such an official document should contain at least the following entries: an entry identifying the dosage form of the medical drug, an entry identifying the dosage regimen of the medical drug, an entry identifying the method of administration of the medical drug. In addition, for example, but not limited to, the entries may relate to a food, such as, for example, a dietary supplement, instead of a medical drug, and therefore, accordingly, the source of official information can be an instruction to a dietary supplement, which contains at least the following entries: an entry identifying the dosage form of the dietary supplement, an entry identifying the dosage regimen of the dietary supplement, an entry identifying the method of administration of the dietary supplement. Furthermore, but not limited to, depending on the function of the implementation employing method 300, the source of official information about the medical drug (dietary supplement) may be required to contain other entries that have to be extracted and cleaned and/or semantically and syntactically analyzed, such as, for example, but not limited to, an entry identifying the active ingredient, an entry identifying effects on driving and operating machinery, an entry identifying interactions with other drugs or substances, etc.

For example, but not limited to, GDMD of ORVIS® Immuno, 125 mg (corresponding to an expert review No. 20438 dated Sep. 24, 2024, URL: https://web.archive.org/web/20250418211233/https://portal.eaeunion.org/sites/ commonprocesses/ru-ru/_vti_bin/Portal.EEC.CDBProxy/Data.svc/GetDocumentFileWithName/ P.MM.01/drug-registrations/668e4e6830dcf88d7dd87397?filePath=ef42c453-4927-4049-877c-0df32c7de366/84f9a38b-eb4e-413f-ad8d-5dbde348348b/ file_13033324029077823.pdf&fileName=02001_1-3-1-proekt-ohlp%20(1616482301).pdf) contains at least the following uncleaned entries identifying its dosage form: in section 1, “MEDICAL DRUG NAME”: “ORVIS Immuno, 125 mg, film-coated tablets”; in section 3, “DOSAGE FORM”: “Film-coated tablets. Shape: round, biconvex, orange film coating. Core: orange, with white and orange flecks.” For example, but not limited to, in the present disclosure, the cleaning of an entry identifying the dosage form of the medical drug involves removing extraneous information from the entry so as to at least allow the medical drugs to be classified by dosage form, and/or to make the notification text for the user as short as possible. For example, but not limited to, after an entry identifying the dosage forms of the medical drug has been cleaned, the resulting entry may contain only “tablets” since the specific implementation may require neither the strength of the active ingredient of the tablet nor its appearance; thus, the cleaned entry (“tablets”) can subsequently be used to quickly search for the medical drug stored in device 100 within the corresponding electronic catalog implemented in device 100 and/or user device 202 and/or server 201; and thus, the cleaned entry (“tablets”) can subsequently be used, for example, but not limited to, to generate a notification to be shown (rendered) on the display or other output means of device 202, which, after being generated and provided with additional information for the user, may thus contain the phrase “It's time to take your pill”. For example, but not limited to, the above GDMD contains at least the following unprocessed entry identifying the dosage regimen of the medical drug: in section 4.2, “Dosage and Administration”: “Dosage. For the treatment of flu and other viral respiratory infections: Initiate therapy at 125 mg once daily. After two days, 125 mg once every 48 hours. Full course: 750 mg (6 tablets). For the prevention of flu and other viral respiratory infections: 125 mg once weekly, for 6 weeks. Full course: 750 mg (6 tablets). For the treatment of a herpes infection: Initiate therapy at 125 mg once daily. After two days, 125 mg once every 48 hours. Course dosage: 1.25-2.5 g (10-20 tablets). Pediatric use. ORVIS Immuno should not be prescribed (administered) to children between 0 and 18 years as it is impossible to establish the dosage regimen.” For example, but not limited to, the semantic and syntactic analysis involves parsing a text in natural language, which at least allows to obtain the necessary statements, such as, but not limited to, the following: an entry identifying the maximum recommended single dose of the medical drug, an entry identifying the maximum recommended daily dose of the medical drug, an entry identifying the standard course of administration of the medical drug. In addition, but not limited to, such entries may not be directly contained in the original unprocessed entry, so that in order to obtain them, various suitable production rules used to provide the required entries can be applied in addition to the semantic and syntactic analysis. For instance, but not limited to, the following entries can be obtained in this way: “Maximum recommended single dose—125 mg”, “Maximum recommended daily dose—125 mg”, “For the treatment of flu and other viral respiratory infections—standard course—10 days”, “For the prevention of flu and other viral respiratory infections—standard course—6 weeks”, “For the treatment of a herpes infection—standard course—18-36 days”. The resulting entries can then be cleaned to obtain, for example, entries identifying the indications of the medical drug, whereupon the electronic catalog for this medical drug can be supplemented with the dosage regimen corresponding to the indications to be selected by the user. For example, but not limited to, depending on the complexity of the dosage regimen, additional entries can be obtained that define the actual administration regimen of the medical drug. For example, but not limited to, in the given case, additional information may be obtained that the initial dose is to be taken once daily for two days, i.e., one tablet every 24 hours, and the subsequent doses are to be taken once every 48 hours. Such information, for example, but not limited to, can subsequently be used to generate instructions regulating the sequence of generating the control signals for unlocking the locking mechanism of device 100 at given times and/or of generating the notifications to be displayed on user device 202 at given times. For example, but not limited to, the above GDMD contains at least the following unprocessed entry identifying the method of administration of the medical drug: in section 4.2, “Dosage and Administration”: “Administration information. Take the preparation orally, after the meal.” For example, but not limited to, a semantic and syntactic analysis of the unprocessed entry above may result in an entry that identifies the method of administration of the medical drug as “orally”. For example, but not limited to, a semantic and syntactic analysis of the unprocessed entry above may result in an entry that identifies the administration of the medical drug relative to meals as “after the meal”. The resulting entries can subsequently be used, for example, but not limited to, to generate a notification to be shown (rendered) on the display or other output means of device 202, which, after being generated and provided with additional information for the user, may thus contain the phrase “It's time to take your pill. Orally. After the meal.” For example, but not limited to, the user may provide their standard meal times and then select a specific meal, when the notification has to be generated and access to the dosage form stored in device 100 has to be provided, wherein the entry “After the meal” can be used to set the time for displaying (rendering) the notification and/or for generating the control signal for unlocking the locking mechanism of device 100. For example, but not limited to, other uncleaned and/or unprocessed entries can also be obtained from each source of official information about the medical drug, such as, for example, but not limited to, an unprocessed entry identifying the contraindications of the medical drug. For example, but not limited to, the above GDMD contains the following unprocessed entry identifying the contraindications of the medical drug: in section 4.3, “Contraindications”: “⋅Hypersensitivity to tilorone or any additional components listed in section 6.1. ⋅ Pregnancy and lactation. ⋅ Children under 18 years.” For example, but not limited to, a semantic and syntactic analysis of the unprocessed entry above may result in an entry that identifies the contraindications for specific populations as “Children under 18 years” and entries that identify the contraindications for specific body statuses as “Hypersensitivity to tilorone”, “Pregnancy”, “Lactation”. For example, but not limited to, the user may provide information about their age and body status, which can be used as a basis for checking the possibility of access to the dosage form stored in device 100, or, for example, display a notification containing a warning about the use of the drug. For example, but not limited to, the above GDMD contains the following uncleaned entry identifying the active ingredient of the medical drug: in section 2, “DESCRIPTION”: “Active ingredient: tilorone. One dosage strength of one film-coated tablet contains 125 mg of tilorone dihydrochloride (tilorone). Notable inactive ingredients contained in the preparation: Sunset yellow colorant (see sections 4.3, 4.4). The full list of inactive ingredients can be found in section 6.1.” For example, but not limited to, the resulting cleaned entry may identify the active ingredient as “tilorone”. For example, but not limited to, such entry can be added to the electronic catalog to enable a quick search for similar medical drugs by the active ingredient, and also, for example, but not limited to, it can be used for associating the database, to which this cleaned entry belongs, with the unique identifiers of other sources of official information about the medical drug that, for some reason, could not be processed when the database was created using method 300 because they were absent.

For example, but not limited to, thanks to the database generated using method 300, it is possible to generate control signals for device 100 for storing and distributing dosage forms based on prescribing information, which facilitates the interaction between the user and device 100. For example, but not limited to, at least one instruction to generate control signals for device 100 can be obtained by means of a method for generating instructions to generate control signals for device 100, the method executed by a processor of a computer device and comprising at least the following steps: obtaining an image of the front side of the prescribing information about the medical drug using the video camera of the user device; recognizing at least one unique property printed on the front side of the prescribing information about the medical drug; receiving at least one dataset associated with an identified unique property to be used to generate said instruction to generate control signals; generating, based on the received dataset, at least one instruction to generate control signals. For example, but not limited to, the user device can be user device 202, which is therefore equipped with a video camera 2026; in addition, for example, but not limited to, user 202 is most typically a smartphone, a PDA, a tablet, etc.

For example, but not limited to, a unique property to be recognized on the front side of a prescribing information can be at least one of a bar code, a medical drug name, a brand name, an active ingredient, or any suitable combination thereof; in addition, for example, but not limited to, the unique property can therefore be associated with another property stored in the database generated using method 300, and, for example, but not limited to, can therefore be used to obtain a dataset based on the obtained (recognized) unique property; in addition, for example, but not limited to, a bar code is a 1D bar code or a 2D bar code, wherein, for example, but not limited to, a 2D bar code is a multilevel 2D bar code or a Data Matrix bar code, which is known in the art at the time of this disclosure or will be known in the future; in addition, for example, but not limited to, the name of the medical drug and/or its brand name and active ingredient can be located inside the same segment of the front side of the prescribing information and be represented by entries in natural language.

For example, but not limited to, based on said instruction, a control signal for unlocking the locking mechanism of device 100 can be generated, wherein device 100 stores in its memory a program code which, when executed by the processor, allows to obtain at least one generated control signal for unlocking the locking mechanism of said device for storing and distributing dosage forms. For example, but not limited to, said control signal can therefore be used to distribute the dosage forms stored in device 100. For example, but not limited to, based on the dataset obtained by recognizing a unique property on the front side of the prescribing information, a notification associated with at least the date and/or time can be generated, which can therefore be displayed by means of the output device of the user device, such as, for example, but not limited to, a display, including a touchscreen of user device 202. For example, but not limited to, the display of the generated notification can be associated with the generation of the control signal for unlocking the locking mechanism of device 100, for example, in such a way that the same program code that prompts the processor of the user device to display said notification also causes the instruction to generate control signals for unlocking the locking mechanism of device 100 to be generated by means of server 201 of system 200 or the user device itself and then sent to device 100 to be used in accordance with the instruction for responding to a received control signal, stored in the memory of device 100; in addition, for example, but not limited to, a notification to take the medical drug after a certain period of time can be shown to the user, wherein, device 100 is therefore provided with a corresponding instruction to generate the control signal, for example, but not limited to, at the end of said period of time.

Thus, for example, but not limited to, there may be provided a method for distributing dosage forms, the method executed by a processor of a computer device and comprising at least the following steps: receiving at least one dataset associated with an identified unique property to be used to generate an instruction to generate control signals; generating, based on the received dataset, at least one instruction to generate control signals; generating, based on the at least one generated instruction, at least one control signal for unlocking the locking mechanism of device 100; and using the generated control signal to unlock the locking mechanism of device 100.

For example, but not limited to, a wearable user device 205 that can be used to identify the body status may be additionally provided. For example, but not limited to, such wearable user device 205 comprises processor 2051 of the wearable user device, measuring device 2052, at least adapted to measure some parameter of the body over a set time period, transceiver 2053 of the wearable user device, memory 2054 of the wearable user device. For example, but not limited to, such wearable user device 205 can be implemented as a smartwatch, a smart ring, a smart bracelet, a fitness tracker, a pulsimeter, a heart rate monitor, etc.; in addition, for example, but not limited to, in this case, device 205 can be additionally equipped with an input device 2055, which may also be an input/output device 2055 represented by, depending on the implementation, for example, but not limited to, a touchscreen, a sensor bracelet, a sensor rim, which allow the user to input commands and instructions to generate various control signal and, if applicable, provide a graphical user interface to facilitate the input of commands and instructions for the user and to render the results of the inputted commands and instructions; in addition, but not limited to, in case device 2055 does not provide output, information can be shown using the display of user device 202 coupled to device 205; in addition, but not limited to, user device 202 can be also equipped with a similar graphical user interface. For example, but not limited to, network 204 can be used to connect device 205 to both device 202 and server 201 of system 200. For example, but not limited to, memory 2054 is identical to memory 2012, 2022 and is therefore not described in any further detail. For example, but not limited to, device 205 can also be equipped with some means for attaching the device to the body, specifically, depending on the implementation of device 205, most typically, but not limited to, a smartwatch strap, a smart bracelet or a fitness tracker bracelet, a ring rim, a pulsimeter or a heart rate monitor strap. For example, but not limited to, measuring device 2052, most typically, is a pulsimeter adapted to, using a conventional photoplethysmography technology, which is not described any further, measure at least the “heart rate” (HR) parameter of the body over a set period of time; in addition, for example, but not limited to, the “heart rate variability” (HRV) parameter can be obtained through HR-based calculations using conventional techniques. For example, but not limited to, measuring device 2052 can be implemented as a transdermal sensor capable of obtaining body parameters related to presence of molecules of a certain substance in the sweat fluids of the body within a set period of time; for example, but not limited to, such transdermal sensor can be implemented in device 2052, for example, as shown in the article “Smartwatch-style sensor could help people avoid excessive drinking” (URL: https://prevention.psu.edu/news/smartwatch-style-sensor-could-help-people-avoid-excessive-drinking/), i.e. in the housing of device 2052, where the photoplethysmographic pulsimeter is most typically situated, or, for example, but not limited to, in means 2056 for attaching device to the body, wherein the sensor uses the Surface-Enhanced Raman Scattering (SERS) technology, for example, using a transdermal sensor disclosed in patent U.S. Pat. No. 12,140,546B2; in addition, for example, but not limited to, the substance in question can be set by the user and/or attending physician in advance or it can be determined based on at least one medical drug taken by the user, which can be obtained based on the datasets related to the sources of official information as was shown above in more detail. For example, but not limited to, such body parameters can be used to obtain datasets, which at least include a sequence of measurements of a parameter of the body over a set period of time. For example, but not limited to, device 205 can be further calibrated, wherein the body parameters are obtained specifically when the body status corresponds to a selected one; for instance, but not limited to, the user may select the “meal: coffee” body status in advance, after which they will drink a certain amount of coffee during a given period of time, which most typically corresponds, for example, to an amount of coffee that the user usually drinks in the morning, wherein device 205 will take a sequence of measurements during this period of time, based on which a dataset associated with the “meal: coffee” body status will be generated. For example, but not limited to, the user may select the “physical activity: strength training” body status in advance, after which they will perform certain exercises or physical labour during a given period of time, which most typically corresponds to the exercises or physical labour usually performed by the user, wherein device 205 will take a sequence of measurements during this period of time, based on which a dataset associated with the “physical activity: strength training” body status will be generated. For example, but not limited to, the user may select the “drug: ivabradine” body status in advance, after which they will take a certain dose of ivabradine during a given period of time, which most typically corresponds to the dose usually taken by the user, wherein device 205 will take a sequence of measurements during this period of time, based on which a dataset associated with the “drug: ivabradine” body status will be generated. In addition, but not limited to, a plurality of parameter sets associated with corresponding body statuses is provided that are used to generate a body status database stored in the memory, which can subsequently be used by a suitable machine learning model to accurately identify the status of the body when device 205 is used. Furthermore, for example, but not limited to, the obtained parameter of the body is, most typically, sent to user device 202 coupled to wearable user device 205, after which the status of the body is identified and/or the parameter of the body is forwarded to server 201 of system 200 in order to identify the status of the body using the capabilities of server 201; alternatively, for example, but not limited to, the parameter of the body may be immediately sent to server 201 or to device 100, 202. Furthermore, for example, but not limited to, the obtained parameter of the body can be used to generate at least one instruction to generate a control signal for device 100, wherein said control signal can be generated, for example, upon identifying a specific body status or, for example, in case the identified body status is different from the previously identified one, i.e., for example, but not limited to, when the “meal: coffee” body status is identified, a control signal can be generated for device 100 to distribute the dosage form of the drug that has a beneficial interaction with caffeine, such as, for example, but not limited to, vitamin B12, which can be more easily digested thanks to increased gastric acid production; in addition, for example, but not limited to, a notification can be shown to the user, for example, containing the phrase: “You have drunk coffee. Do not forget to take vitamin B12 within 15 minutes.” For example, but not limited to, it may be determined in advance that iron preparations have been prescribed to the user, and therefore, in case the “meal: coffee” body status has been identified, access to the dosage form of the iron preparation stored in device 100 may be blocked until another body status is identified, which is different “meal: coffee” and does not prevent the user from taking iron preparations, and in this case, but not limited to, a notification can be shown to the user, for example, containing the phrase: “You have drunk coffee. Do not take iron preparations for at least 40 minutes”; and after a certain time has elapsed or when a new body status is identified, which shows the absence of polyphenols and tannins in the body at least in concentrations that prevent the absorption of iron, the following notification may be generated: “You can take iron preparations”; in addition, an instruction may be provided to generate a corresponding control signal for device 100 to distribute the dosage form.

The present disclosure of the proposed invention demonstrates only certain exemplary embodiments of the invention, which by no means limit the scope of the claimed invention, meaning that it may be embodied in alternative forms that do not go beyond the scope of the present disclosure and which may be obvious to persons having ordinary skill in the art.

1. A method for generating a database of body statuses, the method executed by a processor of a computer device and comprising at least the following steps:
at least by means of a graphical user interface of a user device, displaying a list of body statuses corresponding to one of a meal, physical activity, drug, or any suitable combination thereof;
at least by means of a graphical user interface of a user device, selecting one body status from the list;
by means of a wearable user device, obtaining at least one parameter of a body, namely one of a heart rate over a set period of time, a heart rate variability over a set period of time, presence of molecules of a certain substance in sweat fluids of a body within a set period of time, or any suitable combination thereof,
wherein the parameter of the body is obtained when its body status corresponds to the selected one;
based on the obtained parameter of the body, generating a dataset associated with the selected body status,
wherein the dataset includes at least a sequence of measurements of the parameter of the body over a set period of time;
generating a database of body statuses that includes at least one dataset associated with the selected body status;
wherein a user device comprises at least:
a processor of the user device;
one of an input/output device of the user device or a combination of an input device of the user device and an output device of the user device;
a transceiver of the user device;
a memory of the user device that stores at least:
a program code, which, when executed by the processor of the user device, causes a graphical user interface of the user device to be generated, at least adapted to render the list of body statuses and to select one body status from the list,
and a program code, which, when executed by the processor of the user device, causes the transceiver of the user device to receive the obtained parameter of the body;
wherein a wearable user device comprises at least:
a processor of the wearable user device;
a measuring device, at least adapted to measure some parameter of the body during a set time period;
a transceiver of the wearable user device;
a memory of the wearable user device that stores at least:
a program code, which, when executed by the processor of the wearable user device, causes the at least one parameter of the body to be obtained,
and a program code, which, when executed by the processor of the wearable user device, causes the transceiver of the wearable user device to send the obtained parameter of the body.

2. The method of claim 1, characterized in that the measuring device is one of a pulsimeter, a transdermal sensor, or any suitable combination thereof.

3. The method of claim 1, characterized in that the dataset is used for generating at least one instruction, according to which at least one control signal for unlocking a locking mechanism of a device for storing and distributing dosage forms is generated, wherein the device for storing and distributing dosage forms comprises at least:
a compartment for storing a dosage form adapted to hold dosage forms in their original packaging, wherein the compartment is equipped with a processor-controlled locking mechanism;
a processor adapted to control the locking mechanism;
a transceiver;
a memory that stores at least:
a program code, which, when executed, by a processor, causes at least one instruction to generate control signals for unlocking the locking mechanism to be received,
and a program code, which, when executed by a processor, prompts it to generate a control signal for unlocking the locking mechanism in accordance with the at least one received instruction,
wherein such instruction is generated using at least one dataset contained in the database generated using the above method for generating a database, performed by a processor of a computer device.

4. The method of claim 3, characterized in that the compartment is configured to hold dosage forms in their original packaging, such as one of the following: an ampoule, a jar, a bottle, a syringe, a box, a packet, a sachet, a tube, a contour bagless pack, a contour cellular (blister) pack, a vial, or a syringe tube.

5. The method of claim 3, characterized in that said instruction is received from a server of a system for distributing dosage forms.

6. The method of claim 3, characterized in that the memory additionally stores a program code, which, when executed by the processor, ensures generation and sending of a signal for unlocking the compartment.

7. The method of claim 3, characterized in that the compartment is equipped with at least one sensor adapted to detect the fact that the original packaging has been removed from the compartment.

8. The method of claim 3, characterized in that the compartment is equipped with at least one sensor adapted to detect the fact that the original packaging has been placed into the compartment.

9. The method of claim 8, characterized in that the memory additionally stores a program code, which, when executed by a processor, prompts it to generate a control signal for locking the locking mechanism.

10. The method of claim 9, characterized in that the control signal for locking the locking mechanism is generated in response to a detected fact of placing an original packaging into the compartment.
